(19) United States
(12) Patent Application Publication
(10) Pub. No.: US 2026/0166328 A1
(43) Pub. Date: Jun. 18, 2026

(54) Implantable Cardioverter-Defibrillator

(71) Applicant: BIOTRONIK SE & Co. KG, Berlin (DE)

(72) Inventors: Thomas DOERR, Berlin (DE); Ingo WEISS, Pforzheim (DE)

(73) Assignee: BIOTRONIK SE & Co. KG, Berlin (DE)

(21) Appl. No.: 19/126,933

(22) PCT Filed: Oct. 9, 2023
(86) PCT No.: PCT/EP2023/077885

§ 371 Date: May 2, 2025

(30) Foreign Application Priority Data
Nov. 9, 2022 (EP) 22206399.2

(51) Int. Cl. A61N 1/39

(52) U.S. Cl. CPC A61N 1/3956; A61N 1/3968

(57) ABSTRACT

An implantable cardioverter-defibrillator including: a housing, wherein the housing includes a stimulation unit and a sensing unit; a combined defibrillation and sensing electrode for delivering stimulation pulses to a patient in need thereof and for sensing electrocardiogram signals of the same patient, wherein the combined defibrillation and sensing electrode is electrically connected to the stimulation unit and to the sensing unit. The combined defibrillation and sensing electrode includes an electrode pole that serves for delivering stimulation pulses to a patient in need thereof and for sensing electrocardiogram signals of the same patient. In addition, the sensing unit includes a circuitry enabling the sensing unit to tolerate a post-potential of a stimulation pulse delivered by the combined defibrillation and sensing electrode.

### CROSS-REFERENCE TO RELATED APPLICATIONS

This application is the United States National Phase under 35 U.S.C. § 371 of PCT International Patent Application No. PCT/EP2023/077885, filed on Oct. 9, 2023, which claims the benefit of European Patent Application No. 22206399.2, filed on Nov. 9, 2022, the disclosures of which are hereby incorporated by reference herein in their entireties.

### TECHNICAL FIELD

The present invention relates to an implantable cardioverter-defibrillator according to the preamble of claim 1 and to a method for operating such an implantable cardioverter-defibrillator according to the preamble of claim 13.

### BACKGROUND

In implantable cardioverter-defibrillator (ICD) systems known from prior art electrode poles used for delivering a defibrillation shock in a high-energy therapy cannot be used for sensing electrocardiogram signals since they are biased with electric post-potentials that overmodulate sensitive sensing amplifiers typically implemented within ICD systems. Thus, typically multipolar electrode leads are used to provide a distinct sensing vector for sensing electrocardiogram signals after a defibrillation shock or during or after a post-shock stimulation. However, there is a need for less complex electrode leads and connector systems which are less prone to error and cheaper in production.

Thus, quite complex electrode leads and connector systems for connecting typically at least three electrode poles are necessary according to prior art solutions. This results in a quite thick electrode lead that is prone to error and expensive in production.

The present disclosure is directed toward overcoming one or more of the above-mentioned problems, though not necessarily limited to embodiments that do.

### SUMMARY

It is an object of the present invention to provide an ICD having a less complex construction of its electrode and an improved sensing behavior of electrocardiogram signals comparable with prior art ICDs having a complex electrode construction. It is a further object of the present invention to provide a method for operating such an ICD.

These objects are achieved with an implantable cardioverter-defibrillator (ICD) having the features of claim 1 and a method for operating an ICD having the features of claim 13.

According to an aspect of the present invention, such an ICD comprises a housing that-in turn comprises a stimulation unit and a sensing unit. The ICD furthermore comprises a combined defibrillation and sensing electrode that serves for delivering stimulation pulses to a patient in need thereof and for sensing electrocardiogram signals of the same patient. In this context, the combined defibrillation and sensing electrode is electrically connected to the stimulation unit and to the sensing unit.

According to an advantageous embodiment of the present invention, the implantable ICD may be a non-transvenous ICD. The term “non-transvenous” in this respect in particular shall express that the electrode of the non-transvenous ICD does not extend transvenously into the patient's heart, but fully rests outside of the patient's heart. Expressed in other words, the non-transvenous ICD is configured for non-transvenous implantation, i.e., for an implantation such that no electrode leads are implanted transvenously within the heart of a (human or animal) patient. The non-transvenous ICD hence is to be implanted in a patient such that a generator and an electrode arrangement, for example a shock electrode placed on a lead connected to the generator, are implanted extracardially and do not reach into the heart of the patient, that is into the right or left ventricle or the right or left atrium.

In an embodiment, the combined defibrillation and sensing electrode is a non-transvenous, in particular a subcutaneously, a submuscularly and/or a substernally implantable electrode. The ICD in particular is designed for emitting electrical shocks in case life-threatening arrhythmias of a patient's heart are detected. By means of an electrical shock, a defibrillation shall be achieved in order to reset the cardiac rhythm back to a normal state.

The combined defibrillation and sensing electrode is, in an implanted state of the non-transvenous ICD, placed outside of the patient's heart, e.g., in the region of the sternum of the patient, such that a shock pulse for achieving a defibrillation is generated outside of the heart. Generally, the non-transvenous ICD does not comprise any portions that extend transvenously into the heart, but the defibrillator device is configured to achieve a sensing and emission of signals outside of the heart.

According to a further embodiment of the present invention, the combined defibrillation and sensing electrode may comprise only a single electrode pole. This single electrode pole is arranged and designed for delivering stimulation pulses to a patient in need thereof and for sensing electrocardiogram signals of the same patient. Since only a single electrode pole is used, only a single electrode lead is necessary to electrically contact this single electrode pole. This significantly reduces the complexity of the combined defibrillation and sensing electrode so that it can be manufactured in a cheaper way than electrodes known from prior art. Furthermore, it can be dimensioned much thinner than electrodes known from prior art and is less prone to errors since it comprises less electrode leads that could break during operation of the ICD.

The sensing unit of the ICD furthermore comprises a circuitry that enables the sensing unit to tolerate a post-potential of a stimulation pulse delivered by the combined defibrillation and sensing electrode. By such additional circuitry, an overmodulation of the sensitive sensing amplifier of the sensing unit is avoided. Thus, the combination of a less complex construction of the combined defibrillation and sensing electrode and a sensing unit having higher tolerances with respect to the provided input signals results in an ICD having a much less complex construction than ICDs known from prior art and allowing for a cost reduction of its manufacturing process and a reduction of possible complications such as electrode breakages or product recalls due to anticipated electrode breakages.

In an embodiment, the single electrode pole of the combined defibrillation and sensing electrode may be designed as shock coil. Such a shock coil is particularly appropriate for delivering defibrillation shocks as stimulation pulses. In addition, a shock coil is likewise appropriate for delivering a post-shock stimulation and for sensing electrocardiogram signals of the patient.

In an embodiment, the single electrode pole of the combined defibrillation and sensing electrode is designed such to have the same dimensions as an electrode lead body that is used to house an electrode lead connecting the single electrode pole with the stimulation unit and the sensing unit. By such an isodiametric construction of the single electrode pole with respect to the electrode lead body, a particularly thin and easily implantable defibrillation and sensing electrode can be provided.

In an embodiment, the circuitry provides the sensing unit with a dynamic range that allows the sensing unit to tolerate a post-potential of a stimulation pulse previously delivered by the combined defibrillation and sensing electrode. Such an extended dynamic range with respect to sensing units known from prior art enables the sensing unit to process electrocardiogram signals received with the combined defibrillation and sensing electrode even though the signals are still biased with electric post potentials. Thus, increasing the dynamic range of the sensing unit is a particularly simple, yet effective way to trim the ICD in such a way that a low-complex combined defibrillation and sensing electrode, preferably comprising only a single electrode pole, can be used to operate the ICD.

In an embodiment, the circuitry serves for performing a high pass filtering of signals received by the combined defibrillation and sensing electrode. Such high-pass filtering is a particularly appropriate tool for increasing the dynamic range of the sensing unit while avoiding an overmodulation of the sensing unit due to electric pulses having a particularly high amplitude.

In an embodiment, the circuitry is designed and arranged to perform a direct current (DC) offset compensation of signals received by the combined defibrillation and sensing electrode prior to a processing of the signals by the sensing unit. By such DC offset compensation, the received signals are transformed to a level at which the sensing unit can easily further process the signals. Therefore, such DC offset compensation is also a particularly appropriate measure for preprocessing the received signals to enable the use of a combined defibrillation and sensing electrode, in particular having only a single electrode pole.

In an embodiment, in case of only a single electrode pole, the combined defibrillation and sensing electrode comprises an electrode lead that is used to electrically connect the single electrode pole with the sensing unit and the stimulation unit. This electrode lead is connected with the stimulation unit and/or with the sensing unit with a unipolar connector. In contrast to prior art solutions that require a multipolar connector (such as tripolar connector), the presently implemented combined defibrillation and sensing electrode allows the use of a much less complex connector. This also reduces the costs of manufacturing the ICD as well as its longevity due to a reduced amount of parts that can have failures during their lifetime.

In an embodiment, the ICD comprises a communication unit by which the ICD can be connected with a home monitoring system in a wireless manner. Then, it is possible to monitor the functioning of the ICD from a remote entity and optionally to perform setting adjustments of the ICD from this remote entity. All standard data transmission protocols or specifications are appropriate for such a wireless data communication. Examples of standard data transmission protocols or specifications are the Medical Device Radiocommunications Service (MICS), the Bluetooth Low Energy (BLE) protocol and the Zigbee specification.

Providing an additional circuitry for the sensing unit does not increase the total volume of the ICD or its housing, respectively. In an embodiment, the housing of the ICD has a volume of not more than 70 cm³, in particular of not more than 60 cm³. In an embodiment, the housing has a volume lying in a range of from 40 cm³ to 70 cm³, in particular of from 50 cm³ to 60 cm³.

Since the combined defibrillation and sensing electrode can be designed with a particularly small diameter and since only a unipolar connector is necessary to electrically connect an electrode lead of the combined defibrillation and sensing electrode with the stimulation unit and/or the sensing unit, and electrode connecting area (the so-called header) of the housing can be designed in a particularly small manner. In an embodiment, the header of the housing has a volume of not more than 6 cm³, in particular of not more than 5 cm³. In an embodiment, the header of the housing has a volume lying in a range of from 3 cm³ to 6 cm³, in particular of from 4 cm³ to 5 cm³.

According to a further embodiment of the present invention, the implantable ICD may be a transvenously implantable ICD which means that the combined defibrillation and sensing electrode extends transvenously into the patient's heart. In particular, the transvenously implantable ICD may be implanted such that the combined defibrillation and sensing electrode extends into the right or left ventricle or the right or left atrium.

In an aspect, the present invention relates to a method for operating an ICD according to the preceding explanations. In this context, the method comprises the steps explained in the following.

In a first step, the electrode pole of the combined defibrillation and sensing electrode of the ICD is used to receive electrocardiogram signals of a patient carrying the ICD in an implanted state. Preferably, the housing of the ICD may serve as counter electrode for the combined defibrillation and sensing electrode. However, other kinds of counter electrodes are also possible, for instance a superior-vena-cava (SVC) electrode which may be used in particular in a transvenous ICD.

In a further method step, the received electrocardiogram signals are preprocessed with the circuitry of the sensing unit of the ICD. This preprocessing enables the sensing unit to tolerate a post-potential of a stimulation pulse previously delivered by the combined defibrillation and sensing electrode of the ICD.

Afterwards, the preprocessed signals are processed by the sensing unit.

Thus, in contrast to prior art solutions, preprocessing of the received electrocardiogram signals takes place to allow an easier subsequent processing of the signals by the sensing unit. Due to this preprocessing, it is possible to detect the respective electrocardiogram signals with the electrode pole, that serves both for delivering stimulation pulses to the patient and for sensing electrocardiogram signals of the patient, of the combined defibrillation and sensing electrode despite of the post-potential being present on the electrode pole after a stimulation pulse.

In an embodiment, the preprocessing of the received electrocardiogram signals comprises subjecting the received electrocardiogram signals to a high-pass filter. By such high-pass filter, parts of the received electrocardiogram signals having a particularly high frequency and/or high amplitude are filtered out from the overall received signals. The remaining signals can then be easily processed by the sensing unit without overmodulating the sensing unit.

In an embodiment, the preprocessing of the received electrocardiogram signals comprises applying a DC offset compensation. In doing so, the level of the received electrocardiogram signals is set to values around an average value that can be well sensed by the sensing unit. By such a kind of preprocessing of the received electrocardiogram signals, an overmodulation of the sensing unit can be avoided, thus enabling the sensing unit to process the preprocessed signals in an appropriate way.

In an aspect, the present invention also relates to an implantable medical device for stimulating a body part of a patient, wherein the implantable medical device has the claim elements explained in the following. Such an implantable medical device comprises a housing, wherein the housing in turn-comprises a stimulation unit, sensing unit, and a control unit. The implantable medical device furthermore comprises a combined stimulation and sensing electrode for delivering stimulation pulses to a patient in need thereof and for sensing electric signals of the same patient. In this context, the combined stimulation and sensing electrode is electrically connected to the stimulation unit and to the sensing unit.

According to an aspect of the present invention, the stimulation unit is designed and arranged for generating stimulation pulses and compensation pulses. In this context, the combined stimulation and sensing electrode also serves for delivering compensation pulses to the patient. These compensation pulses do not have a therapeutic effect. Rather, they are intended to reduce a post-potential of the combined stimulation and sensing electrode after having delivered a stimulation pulse. Expressed in other words, the compensation pulses serve for a faster depolarization of the combined stimulation and sensing electrode than in case of self-discharge. To achieve a particularly efficient depolarization, the control unit is designed and arranged for allowing the generation of a compensation pulse by the stimulation unit only with a predetermined temporal delay after a stimulation pulse has been generated by the stimulation unit. This temporal delay can be adjusted to the specific needs of individual embodiments of the implantable medical device. By such an adjustment of the temporal delay, an optimized depolarization of the combined stimulation and sensing electrode can be realized.

An example for an appropriate design and arrangement of the control unit to perform the before-mentioned tasks is to provide an access for the control unit to a memory unit in which a computer-readable code is stored, wherein the computer-readable code causes a processor of the implantable medical device to perform the steps explained in the following when being executed on the processor. In a step, the control unit suppresses the possibility of generating a compensation pulse as long as a predetermined temporal delay after the generation of the stimulation pulse by the stimulation unit has not yet been passed. In a further step, the control unit allows the generation of the compensation pulse by the stimulation unit after the predetermined temporal delay has been passed. The control unit, the sensing unit, and any other unit of the implantable medical device can have a similar design and arrangement to perform the tasks for which it is designed and arranged. Thus, by having access to an appropriate computer program stored within the implantable medical device (in particular within a memory unit of the implantable medical device) and serving for executing certain steps by the respective units, such design and arrangement of the respective units can be particularly easily realized.

The presently claimed and described implantable medical device enables significantly higher performant sensing properties after having used the combined stimulation and sensing electrode for electrotherapeutic applications, in particular for high-energy therapy. It is not necessary to implement additional electrode poles into the electrode. Rather, a particularly simple construction of the combined stimulation and sensing electrode can be realized in the presently claimed and described implantable medical device. The combined stimulation and sensing electrode has, in an embodiment, an “integrated bipolar” configuration in which an electrode pole used for stimulating a body part is also used as one of the electrode poles for sensing electric signals from the body part. To give an example, a shocking coil, which is typically used for delivering stimulation pulses in form of electric shocks like defibrillation shocks, is also used as indifferent electrode pole for a sensing electrode pole such as a tip electrode pole.

In an embodiment, the implantable medical device has a “coil only” configuration in which a shocking coil is used as the only electrode pole of the combined stimulation and sensing electrode. In such a “coil only” configuration, the housing of the implantable medical device serves as counter electrode.

In an embodiment, the compensation pulse has an absolute value of total charge (calculated by the integral of the absolute value of the charge) that is smaller than an absolute value of total charge of the preceding stimulation pulse. Due to this limitation of the total charge of the compensation pulse, an overcompensation of a post-potential of the combined stimulation and sensing electrode is efficiently avoided.

In an embodiment, the compensation pulse has a net charge having a first polarity. Furthermore, the stimulation pulse preceding the compensation pulse has a net charge having a second polarity. In this embodiment, the second polarity is opposite to the first polarity. By such differently polarized pulses, a particularly efficient depolarization of a post-potential of the combined stimulation and sensing electrode can be accomplished with the compensation pulse.

In an embodiment, the predetermined temporal delay lies in a range of from 0.1 ms to 500 ms, in particular from 0.5 ms to 450 ms, in particular from 1 ms to 400 ms, in particular from 2 ms to 350 ms, in particular from 5 ms to 300 ms, in particular from 10 ms to 250 ms, in particular from 20 ms to 200 ms, in particular from 30 ms to 150 ms, in particular from 40 ms to 100 ms, in particular from 50 ms to 90 ms, in particular from 60 ms to 80 ms.

In an embodiment, the stimulation unit comprises a capacitor that is used for generating the compensation pulse. By discharging such a capacitor, the necessary energy for generating the compensation signal can be provided in a very short time and/or a specified range that suits the needs of the compensation pulse.

In an embodiment, the stimulation unit is arranged and designed to generate the compensation pulse at least partially from a residual energy remaining after having generated a stimulation pulse. Upon generating a stimulation pulse, typically, not all energy stored in a capacitor or in another entity of the stimulation unit is in fact needed to generate the stimulation pulse. While the remaining energy could be fed back to the accumulator of the implantable medical device, this would typically result in a significant loss of energy during this process. Therefore, it is more appropriate to otherwise use this residual energy. The generation of the compensation pulse is a particular appropriate measure for operating the implantable medical device in a particularly energy-saving manner.

In an embodiment, the stimulation unit is arranged and designed to generate the compensation pulse partly with an amount of freshly generated charge and partly from the residual energy remaining after having generated a stimulation pulse. The freshly generated additional amount of charge used to generate the compensation pulse accounts for not more than 50% of the total amount of charge of the compensation pulse, in particular for not more than 40%, in particular for not more than 30%, in particular for not more than 20%, in particular for not more than 10%. In an embodiment, the freshly generated additional amount of charge accounts for a value lying in a range of from 5% to 50% of the total amount of charge of the compensation pulse, in particular of 10% to 40%, in particular of 20% to 30%.

In an embodiment, the control unit is designed and arranged to determine a shape, an amplitude, a duration, a net charge, and/or a polarity of the compensation pulse. In this context, the polarity is in particular the polarity of a net charge of the compensation pulse. After such determination by the control unit, the stimulation unit will generate a compensation pulse according to the prerequisites having been defined by the control unit.

In an embodiment, the sensing unit is designed and arranged to determine whether a sensed electric signal lies outside a dynamic range of the sensing unit. If the sensed electric signal lies outside the dynamic range, no evaluation of the electric signal is possible. Rather, it is mandatory that the parameters of the sensed electric signal comply with the dynamic range of the sensing unit to be able to properly evaluate the sensed electric signal. Whenever the sensed electric signal exceeds an upper threshold of the dynamic range of the sensing unit or falls below a lower threshold of the dynamic range of the sensing unit, it lies outside the dynamic range.

Even if the sensed electric signal lies outside the dynamic range of the sensing unit, some basic parameters in relation to the sensed electric signal can be determined nonetheless. In an embodiment, the sensing unit is designed and arranged to determine the time period during which the sensed electric signal lies outside the dynamic range of the sensing unit. Additionally or alternatively, the sensing unit is designed and arranged to determine whether the sensed electric signal exceeds an upper threshold of the dynamic range of the sensing unit or falls below a lower threshold of the dynamic range of the sensing unit. By such determination, the polarity of the sensed electric signal can be determined. If the sensed electric signal lies outside the dynamic range of the sensing unit and exceeds an upper threshold of the sensing unit, it has a positive polarity. If the sensed electric signal, however, lies outside the dynamic range of the sensing unit and falls below a lower threshold of the dynamic range of the sensing unit, it has a negative polarity.

In an embodiment, the control unit is designed and arranged to optimize a success of the generated compensation pulse. In this context, the success is a reduction of the duration during which the electric signals sensed by the sensing unit lie outside the dynamic range of the sensing unit. As will be discussed below, the success can also be defined in a different way. To optimize the success in this embodiment, the time period during which the sensed electric signal lies outside the dynamic range of the sensing unit is evaluated. In addition, an absolute value of the net charge of compensation pulses succeeding a compensation pulse are iteratively increased. By such iterative increment, the effect of the compensation pulses are also iteratively amended. Such iterative increment is performed until one of the following conditions is met. According to a first condition, the sensed electric signal falls below the lower threshold of the dynamic range of the sensing unit if it has exceeded the upper threshold of the dynamic range of the sensing unit in a previous iterative step. Then, the latest iterative step has passed the optimum of the net charge of the compensation pulse. According to a second condition, the sensed electric signal exceeds the upper threshold of the dynamic range of the sensing unit if it fell below the lower threshold of the dynamic range of the sensing unit in a previous iterative step. Also in this case, the latest increment of the compensation pulse has over-optimized the compensation pulse and thus exceeded the optimum range of the net charge of the compensation pulse for achieving a particularly high success with respect to a reduction of the time during which the sensed electric signal lies outside the dynamic range of the sensing unit.

The absolute value of the net charge of the compensation pulse can be increased, e.g., by increasing the amplitude and/or the duration of the compensation pulse and/or by amending the time delay after which the stimulation pulse can be generated. If the time delay is particularly short, the net charge of the compensation pulse is influenced by the preceding stimulation pulse.

In an embodiment, a step size between the individual iterative steps of increasing the absolute value of the net charge of the compensation pulse lies in a range of from 10% to 50%, in particular of from 15% to 40%, in particular from 20% to 30%.

In an embodiment, the step size of the increment of the absolute value of the net charge of the compensation pulse is not identical in the individual iterative steps, but varies at least from some steps to other steps.

In an embodiment, an optimized result for the net charge of the compensation pulse is the average value of the last two to four iterations, in particular the last two to three iterations, in particular the last two iterations, prior to the very last iteration in which the sensed electric signal fell below the lower threshold of the dynamic range of the sensing unit if it has originally exceeded the upper threshold of the dynamic range of the sensing unit or in which the sensed electric signal exceeds the upper threshold of the dynamic range of the sensing unit if it originally fell below the lower threshold of the dynamic range of the sensing unit.

In an embodiment, the sensing unit is designed and arranged to determine a time-dependent change of the post-potential of the sensed electric signal, while the sensed electric signal lies within a dynamic range of the sensing unit. As explained above, a detailed evaluation of the sensed electric signal is only possible if the signal lies within the dynamic range. Therefore, a post-potential and its change over time can be easily determined if the sensed electric signal lies within the dynamic range. The change of the post-potential over time can also be denoted as velocity of depolarization and can be expressed by the change of voltage over time (du/dt).

In an embodiment, the control unit is designed and arranged to optimize a success of the generated compensation pulse by evaluating the time-dependent change of the post-potential of the sensed electric signal and by interactively increasing an absolute value of the net charge of subsequent compensation pulses until the time-dependent change of the post-potential of the sensed electric signal does no longer increase. If the net charge of the compensation pulse is increased to a too high extent, the sign of the change of the post-potential over time changes to the opposite. This is a clear indication that the compensation pulse was increased too much and that a future compensation pulse should be used according to the parameters of the preceding iterative step or of an average value of a plurality of preceding iterative steps.

In this embodiment, the success of the generated compensation pulse is typically also a reduction of the time period during which the sensed electric signal lies outside the dynamic range of the sensing unit. However, this time period is not directly measured. Rather, if the velocity of reduction of the post-potential is high, this is not only valid for the dynamic range of the sensing unit, but typically also for the ranges lying outside the dynamic range of the sensing unit. Thus, if the velocity of reduction of the post-potential is high, this is an indirect measure of a shortening of the time period during which the sensed electric signal lies outside the dynamic range of the sensing unit, even if this time period is not directly determined. Therefore, the success to be achieved according to this embodiment can be seen in increasing the velocity of reduction of the post-potential (i.e., increasing the change of the post-potential over time (du/dt)), wherein the success is an indirect measure of a reduction of the time period during which the sensed electric signal lies outside the dynamic range of the sensing unit.

In an embodiment, a step size between the individual iterative steps of increasing the absolute value of the net charge of the compensation pulse lies in a range of from 10% to 50%, in particular of from 15% to 40%, in particular from 20% to 30%.

In an embodiment, an optimized result for the net charge of the compensation pulse is the average value of the last two to four iterations, in particular the last two to three iterations, in particular the last two iterations, prior to the very last iteration in which the sign of the change of the post-potential over time changes to the opposite.

In an embodiment, the initial value of the iterative steps for increasing the absolute value of the net charge of the compensation pulse is set to a predetermined value by programming the implantable medical device or the control unit, respectively. Such programming can be done with the help of an external device operatively coupled to the implantable medical device.

In an embodiment, the initial value of the iterative steps for increasing the absolute value of the net charge of the compensation pulse are set in dependence on the type of electrode used for the combined stimulation and sensing electrode. In such a case, the characteristic phase boundary capacitance within biological tissue as well as optionally the dependence of the phase boundary capacitance on the therapeutic voltage applied by the stimulation unit can be considered for setting the initial value.

Generally, the implantable medical device can be used to stimulate any body part of a patient in need thereof. To give an example, it can be used as neurostimulation device or as device for stimulating an organ of the patient. In an embodiment, the implantable medical device is designed and arranged for stimulating a human or animal heart and for sensing electric signals from the same heart.

In an embodiment, the implantable medical device is a transvenous implantable cardioverter-defibrillator (ICD). The transvenous ICD is particularly configured such that the sensing of electric signals after having delivered a defibrillation shock or an antitachycardie pacing (ATP) stimulation is carried out with at least one electrode pole that has been previously used for delivering the therapy to the patient.

In an embodiment, the implantable medical device is a non-transvenous ICD. This non-transvenous ICD is particularly configured such that it uses, after having delivered a defibrillation shock, an electrode pole for sensing electric signals, wherein the electrode pole has been previously used for delivering the therapy to the patient. Non-transvenous ICDs having a subcutaneously or a substernally implanted electrode are specific examples of particularly appropriate non-transvenous ICDs.

In an embodiment, the control unit is designed and arranged to cause the stimulation unit to generate a compensation pulse after having delivered a defibrillation shock, after having delivered an antitachycardie pacing, after having delivered at least one pulse during the post shock pacing, and/or after having delivered at least one pulse during a pacing employing signals or pulses having an amplitude of at least 5 V.

In an embodiment, the implantable medical device is compliant with magnetic resonance imaging (MRI) examinations. This facilitates future examinations by MRI of the patient carrying the implantable medical device.

In an embodiment, the implantable medical device comprises a communication unit by which the implantable medical device can be connected with a home monitoring system in a wireless manner. Then, it is possible to monitor the functioning of the implantable medical device from a remote entity and optionally to perform setting adjustments of the implantable medical device from this remote entity. All standard data transmission protocols or specifications are appropriate for such a wireless data communication. Examples of standard data transmission protocols or specifications are the Medical Device Radiocommunications Service (MICS), the Bluetooth Low Energy (BLE) protocol and the Zigbee specification.

In an aspect, the present invention relates to a method for operating an implantable medical device according to the preceding explanations. In this context, the method comprises the steps explained in the following.

In one method step, electric signals of the patient carrying the implantable medical device in an implanted state are received with the combined stimulation and sensing electrode.

Prior to, concomitantly with, and/or after this receiving step, a compensation pulse is generated by a stimulation unit of the implantable medical device with a predetermined temporal delay after the stimulation pulse has been generated by the stimulation unit.

This compensation pulse is then guided to the combined stimulation and sensing electrode to reduce the post-potential of the combined stimulation and sensing electrode after having delivered a stimulation pulse. Due to this reduction of the post-potential, it is much faster possible to fully evaluate the received electric signals of the patient since the electric signals lie much faster within a dynamic range of the sensing unit of the implantable medical device.

In an aspect, the present invention relates to medical method for improving the sensing of electric signals from a patient directly after having delivered a stimulation pulse to the patient. This method comprises the steps explained in the following.

In one method step, a stimulation pulse is generated by a stimulation unit and delivered to a patient with a combined stimulation and sensing electrode. Afterwards, a compensation pulse is generated by the stimulation unit with a predetermined temporal delay after having delivered the stimulation pulse. This compensation pulse is delivered to the patient by the combined stimulation and sensing electrode. However, the compensation pulse has no direct therapeutic effect for the patient. Rather, it serves for reducing the post-potential of the combined stimulation and sensing electrode after having delivered the stimulation pulse.

Prior to, concomitantly with, and/or after having generated and delivered the compensation pulse, electric signals of the patient are received with the combined stimulation and sensing electrode. Due to the effective reduction of the post-potential of the combined stimulation and sensing electrode, this sensing of electric signals can be done in an accurate way much faster than according to prior art techniques that rely on the natural depolarization of the post-potential present on the combined stimulation and sensing electrode.

All embodiments of the ICD can be combined in any desired way and can be transferred either individually or in any arbitrary combination to the implantable medical device, to the implantable cardioverter-defibrillator, and to any of the methods. All embodiments of the implantable medical device can be combined in any desired way and can be transferred either individually or in any arbitrary combination to the non-transvenous ICD, to the implantable cardioverter-defibrillator, and to any of the methods. All embodiments of the implantable cardioverter-defibrillator can be combined in any desired way and can be transferred either individually or in any arbitrary combination to the non-transvenous ICD, to the implantable medical device, and to any of the methods. Likewise, all embodiments of any of the methods can be combined in any desired way and can be transferred either individually or in any arbitrary combination to the non-transvenous ICD, to the implantable medical device, to the implantable cardioverter-defibrillator, and to any of the respective other methods.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further details of aspects of the present invention will be explained in the following making reference to exemplary embodiments and accompanying Figures. In the Figures:

FIG. 1 shows a non-transvenous ICD known from prior art;

FIG. 2A schematically depicts an embodiment of a bipolar non-transvenous ICD;

FIG. 2B schematically shows a typical construction of an embodiment of a non-transvenous ICD;

FIG. 3 illustrates an overmodulation of the sensing unit of a non-transvenous ICD;

FIG. 4 schematically shows a block diagram of an embodiment of a circuitry of a non-transvenous ICD avoiding an overmodulation of the sensing unit as illustrated in FIG. 3;

FIG. 5 schematically shows the temporal development of an electrical signal sensed with a prior art implantable medical device;

FIG. 6 schematically shows a typical construction of an embodiment of an implantable medical device;

FIG. 7 schematically shows the delivery of a stimulation pulse and of a compensation pulse according to an embodiment of the implantable medical device;

FIG. 8A schematically illustrates a first scenario of an embodiment of a reduction of the post-potential of a combined stimulation and sensing electrode;

FIG. 8B schematically illustrates a second scenario of an embodiment of a reduction of the post-potential of a combined stimulation and sensing electrode;

FIG. 8C schematically illustrates a third scenario of an embodiment of a reduction of the post-potential of a combined stimulation and sensing electrode;

FIG. 9A schematically depicts an embodiment of a bipolar transvenous ICD in an implanted state; and

FIG. 9B schematically shows a typical construction of an embodiment of a transvenous ICD.

### DETAILED DESCRIPTION

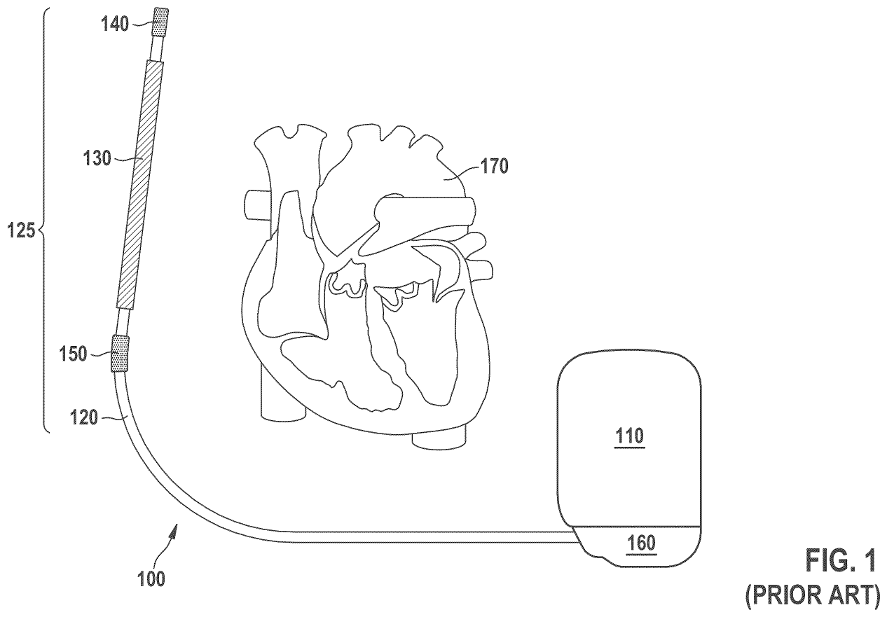

FIG. 1 shows a non-transvenous ICD 100 known from the prior art that comprises a housing 110 and an electrode lead body 120 connected to a sensing unit and a stimulation unit comprised in the housing 110. The electrode lead body 120 forms part of an electrode 125. The electrode 125 comprises a shock coil 130 for delivering defibrillation shocks, a first sensing electrode pole 140 positioned distally of the shock coil 130 at the tip of the electrode 125, and a second sensing electrode pole 150 located proximally of the shock coil 130 and being designed as a ring electrode. The housing 110 serves as counter electrode for the shock coil 130, the first sensing electrode pole 140, and the second sensing electrode pole 150.

The electrode 125 is implanted into human patient in a non-transvenous manner. This is typically done in a left parasternal position or in a sternal position so that the shock coil 130 and the sensing electrode poles 140, 150 are located approximately in a line being parallel to or lying on the sternum of the patient. The electrode 125 is fixed to the tissue of the patient at a position proximally to the second sensing electrode pole 150. The electrode lead 120 is then guided on an angled pathway towards the left side of the patient's thorax and is connected to the sensing unit and the stimulation unit contained in the housing 110 of the non-transvenous ICD 100. This connection is done via a header 160 that needs to be dimensioned such that all electrode leads guided through the electrode lead body 120 to the individual electrode poles 130, 140, 150 have sufficient space.

The position of the shock coil 130 and of the housing 110 result from the anatomic position of the heart 170 of the patient and need to be kept constant for an effective defibrillation of the heart 170.

After having delivered a defibrillation shock by the shock coil 130 or during a post-shock stimulation, the shock coil 130 and the housing 110 serving as counter electrode are highly polarized over a time period lasting for several ten seconds. At least during this time of polarization, the sensing functionality of the ICD 100 is accomplished by the first sensing electrode pole 140 and the second sensing electrode pole 150 that do not form part of the shock path or the post-shock stimulation path.

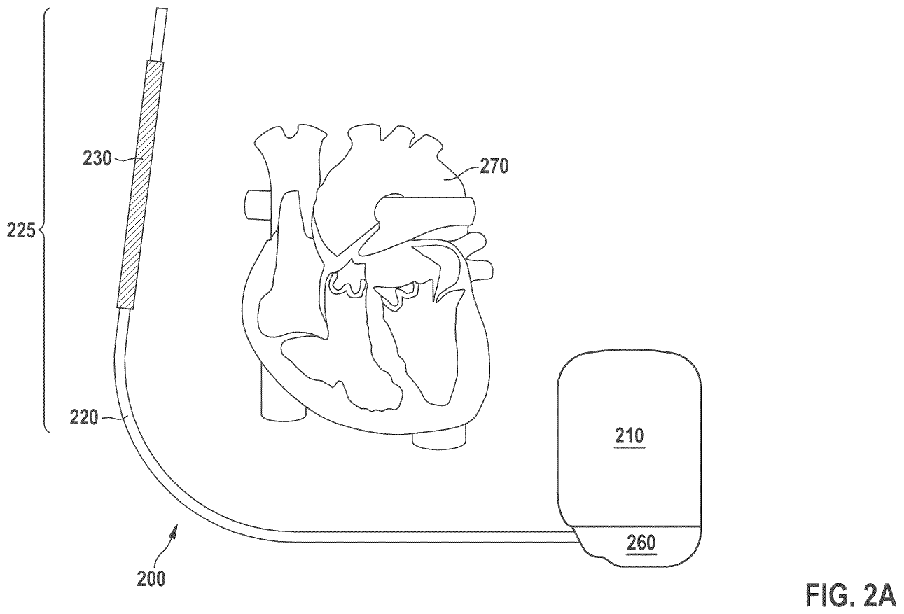

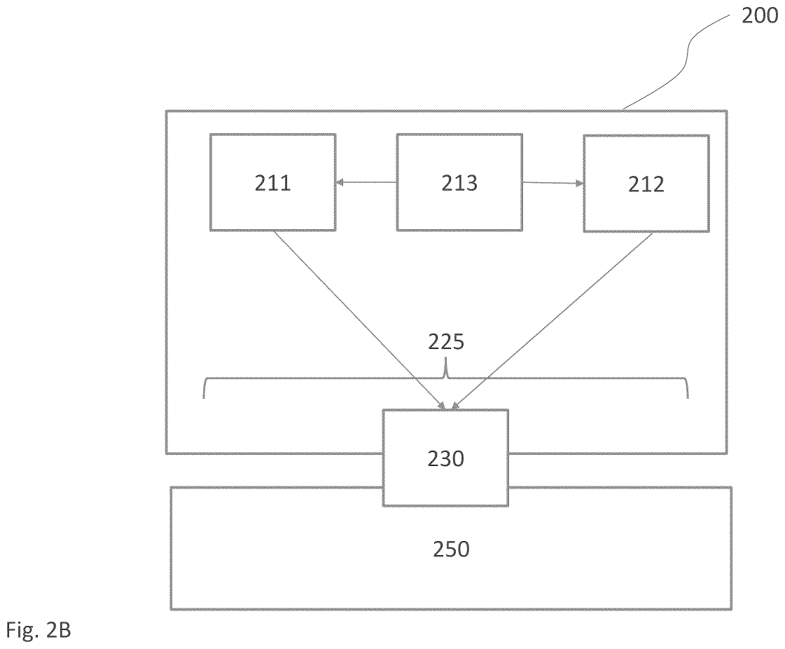

FIG. 2A shows an embodiment of a non-transvenous ICD 200 that is generally implanted in the same way as the ICD shown in FIG. 1. The non-transvenous ICD 200 comprises a housing 210 that contains a sensing unit and a stimulation unit. An electrode lead guided through an electrode lead body 220 is connected to the stimulation unit and the sensing unit. The electrode lead body 220 forms part of an electrode 225 that serves as combined defibrillation and sensing electrode. In contrast to the electrode 125 of the prior art ICD 100 shown in FIG. 1, the electrode 225 of the ICD 200 shown in FIG. 2A comprises only a shock coil 230 as single electrode pole, but no dedicated sensing electrode poles. This shock coil 230 is used for delivering defibrillation shocks to a patient's heart 270 and for sensing electrocardiogram signals from this heart 270. For this purpose, the housing 210 serves as counter electrode for the shock coil 230. The shock coil 230 can also be employed for providing a post-shock stimulation to the heart 270.

Since the electrode 225 comprises only a single electrode pole (namely the shock coil 230) and since it thus only needs to house a single electrode lead, the electrode lead body 220 can be dimensioned smaller than the electrode lead body 120 of the prior art ICD 100 shown in FIG. 1. In addition, a header 260, via which the electrode lead housed within the electrode lead body 220 is electrically connected with the stimulation unit and the sensing unit contained in the housing 210 can be dimensioned smaller than the header 160 of the prior art ICD 100 shown in FIG. 1. This is due to the fact that the electrode 225 can be connected with the sensing unit and the stimulation unit with a unipolar connector that requires less space than a multipolar connector necessary for connecting prior art electrodes.

The sensing unit contained in the housing 210 comprises a circuitry that enables the sensing unit to tolerate a post-potential of a stimulation pulse (such as a defibrillation shock) previously delivered by the electrode 225.

The ICD 200 shown in FIG. 2A can also be denoted as coil-only ICD since the shock coil 230 is the only real electrode pole of this ICD 200. As explained above, the housing 210 serves as counter electrode.

FIG. 2B shows a typical setup of an embodiment of an ICD 200 that can also be denoted as electrotherapeutic implant. The ICD 200 comprises a stimulation unit 211 and a sensing unit 212. Furthermore, it comprises a control unit 230 that is operatively coupled to the stimulation unit 211 and to the sensing unit 212.

The ICD 200 furthermore comprises a shock coil 230 serving as single electrode pole that is located on a combined defibrillation and sensing electrode 225. The shock coil 230 gets into contact with the body of a patient 250. in doing so, the shock coil 230 serves both for delivering a defibrillation shock to the patient 250 as well as for sensing electric signals from the patient 250.

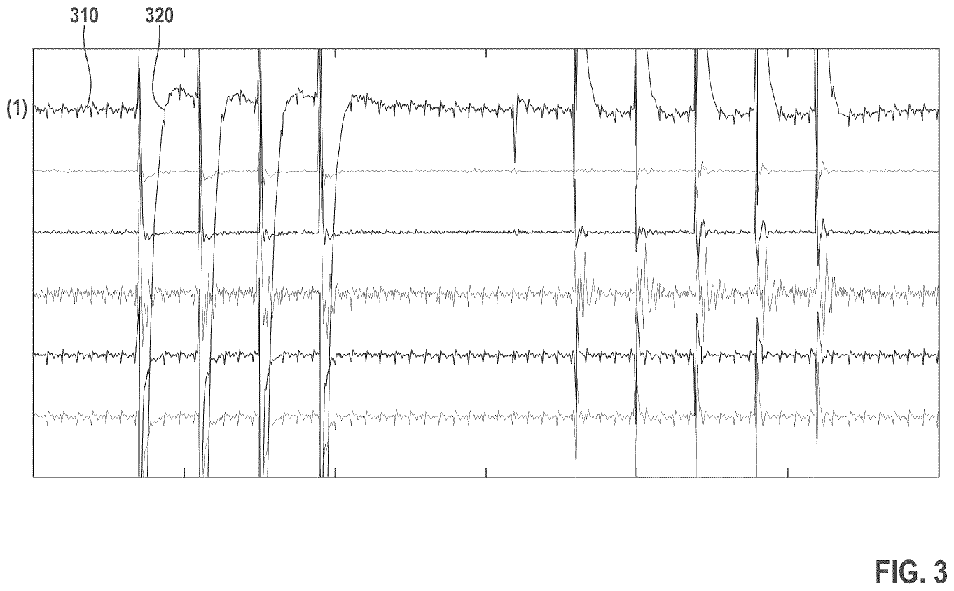

FIG. 3 exemplarily illustrates an overmodulation of the sensing unit due to post-potentials of the electrode poles after having delivered a defibrillation shock or a post-shock stimulation. A regular electrocardiogram (ECG) signal 310 recorded between the shock coil and the housing (cf. curve (1)) of the non-transvenous ICD is overlaid after each stimulation pulse by an artefact signal 320 due to the polarization effects of the electrodes. Regular sensing of the ECG signals is not possible for a plurality of seconds since the input amplifier of the sensing unit is operated outside its dynamic range.

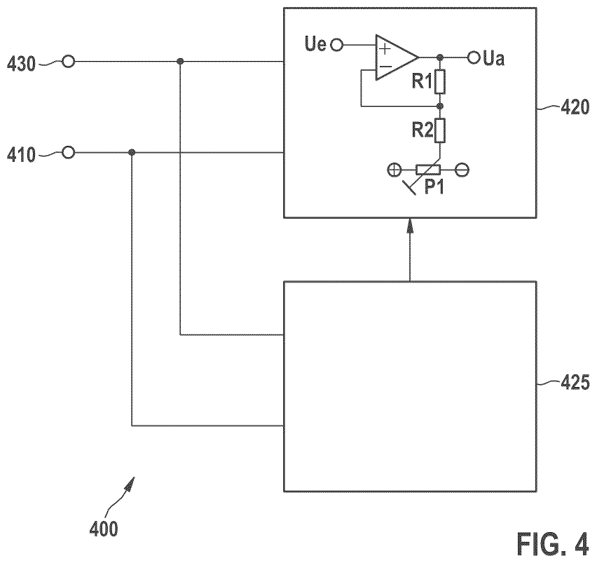

FIG. 4 shows a block diagram of an embodiment of a circuitry 400 that enables the sensing unit of a non-transvenous ICD, e.g., the non-transvenous ICD 200 shown in FIG. 2A, to tolerate a post-potential of a stimulation pulse previously delivered by its electrode. The circuitry 400 comprises a first electrode connector 410 that is intended to be connected with the housing of the ICD, e.g., with the housing 210 of the ICD 200 shown in FIG. 2A. The circuitry 400 further comprises a second electrode connector 430 that is intended to be connected with the stimulation electrode pole such as the shock coil 230 of the ICD 200 shown in FIG. 2A.

The first electrode connector 410 and the second electrode connector 430 are electrically connected with an input amplifier 420. This input amplifier 420 comprises a controllable direct current (DC) offset compensation P1. To control this DC offset compensation P1, the circuitry 400 comprises an offset measuring and controlling unit 425 that is also electrically connected with the first electrode connector 410 and the second electrode connector 430. In addition, it is connected with the DC offset compensation P1 of the input amplifier 420. The offset measuring and control unit 425 measures the DC offset of electrocardiogram signals received after a shock pulse or after a post-shock stimulation, respectively, of the ICD. Then, it controls the DC offset compensation P1 of the input amplifier 420 of the circuitry 400 such that the received signals are brought to a level that can be easily processed by the sensing unit without overmodulating the sensing unit.

It should be noted that this and other embodiments of the non-transvenous ICD can be well combined with a device for compensating polarization artefacts by delivering compensation pulses for actively discharging a polarization after having delivered a shock pulse and/or a post-shock stimulation with an ICD.

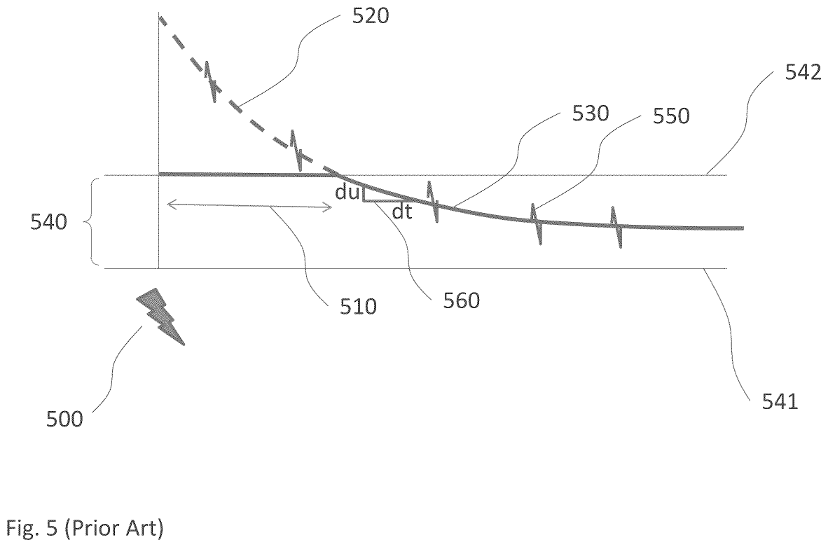

FIG. 5 schematically illustrates the situation according to a prior art solution of an implantable medical device for stimulating a body part and sensing electric signals from the body part. After having applied an electrotherapy with a therapeutic signal 500, it is necessary to wait for a first time period 510 until the polarization or charge on the electrode of the used implantable medical device, which is expressed by a post-potential 520, has been naturally reduced to such an extent that the sensed electric signal 530 lies within a dynamic range 540 of an amplifier of the sensing unit of the implantable medical device. The dynamic range 540 has a lower threshold 541 and an upper threshold 542 that limit the dynamic range 540. During this first time period 510, the sensed electric signal 530 lies constantly on the upper threshold 542 of the dynamic range 540 of the sensing unit. It is not possible to monitor any biologic activities during this first time period 510. Only after the sensed electric signal 530 lies within the dynamic range 540, it is possible to detect small variations of the sensed electric signals, e.g., a cardiac activity 550. A velocity 560 of a change of the post-potential over time (du/dt) is comparatively small. Therefore, the first time period 510, during which no evaluation of the sensed electric signal 530 is possible, is comparatively long.

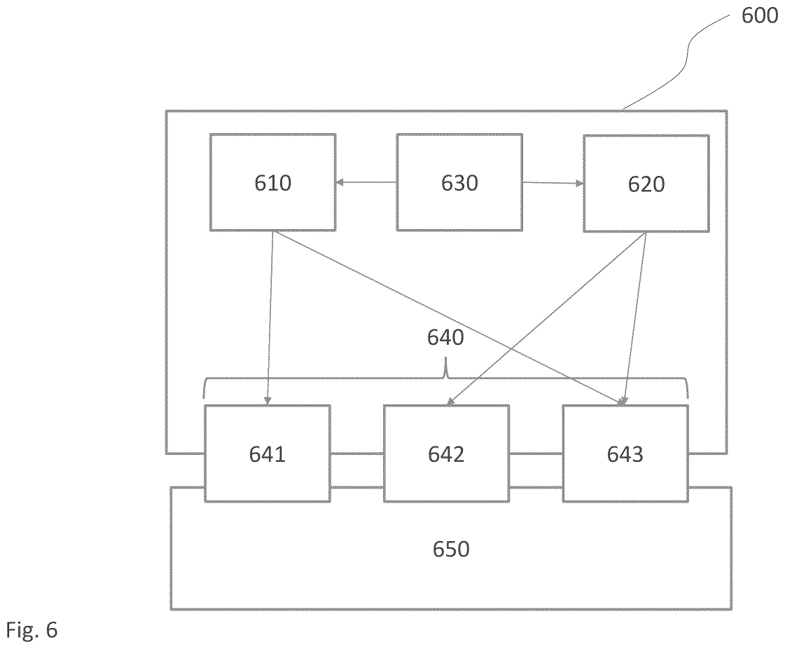

FIG. 6 shows a typical setup of an embodiment of an implantable medical device 600 that can also be denoted as electrotherapeutic implant. An implantable cardioverter-defibrillator (ICD), either transvenous or non-transvenous, is an appropriate example for such an implantable medical device 600. The implantable medical device 600 comprises a stimulation unit 610 and a sensing unit 620. Furthermore, it comprises a control unit 630 that is operatively coupled to the stimulation unit 610 and to the sensing unit 620.

The implantable medical device 600 furthermore comprises a first electrode pole 641, a second electrode pole 642, and a third electrode pole 643 that are located on a combined stimulation and sensing electrode 640. The first electrode pole 641, the second electrode pole 642, and the third electrode pole 643 get into contact with the body of a patient 650. At least one of the electrode poles 641, 642, and 643 serves both for delivering a stimulation pulse to the patient 650 as well as for sensing electric signals from the patient 650. In case of the embodiment shown in FIG. 6, this is true for the third electrode pole 643 that is operatively connected both with the stimulation unit 610 and with the sensing unit 620. The first electrode pole 641 serves as counter electrode pole for the first electrode pole 641 in case of providing a stimulation pulse with the stimulation unit 610, wherein the second electrode pole 642 serves as counter electrode pole for the third electrode pole 643 in case of sensing electric signals with the sensing unit 620.

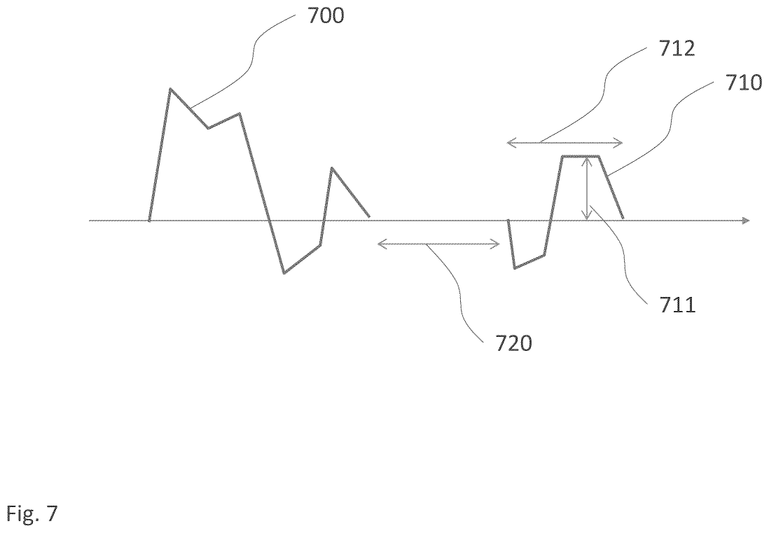

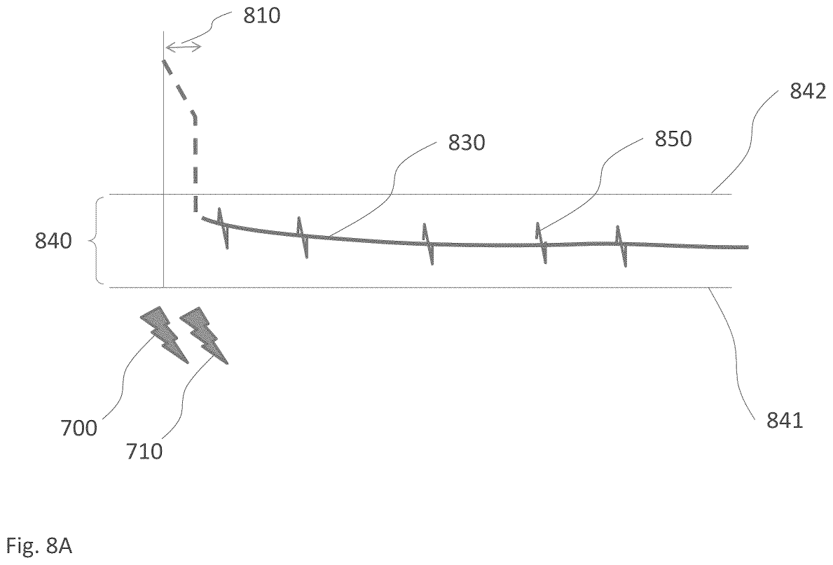

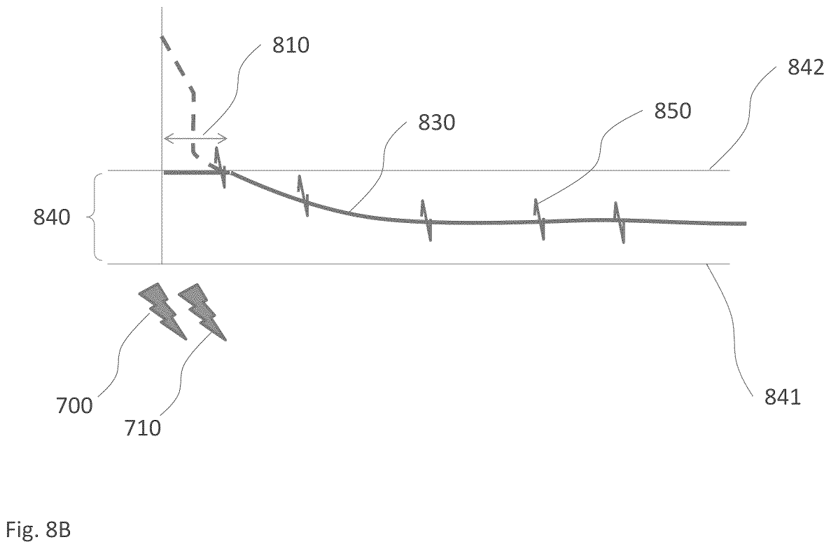

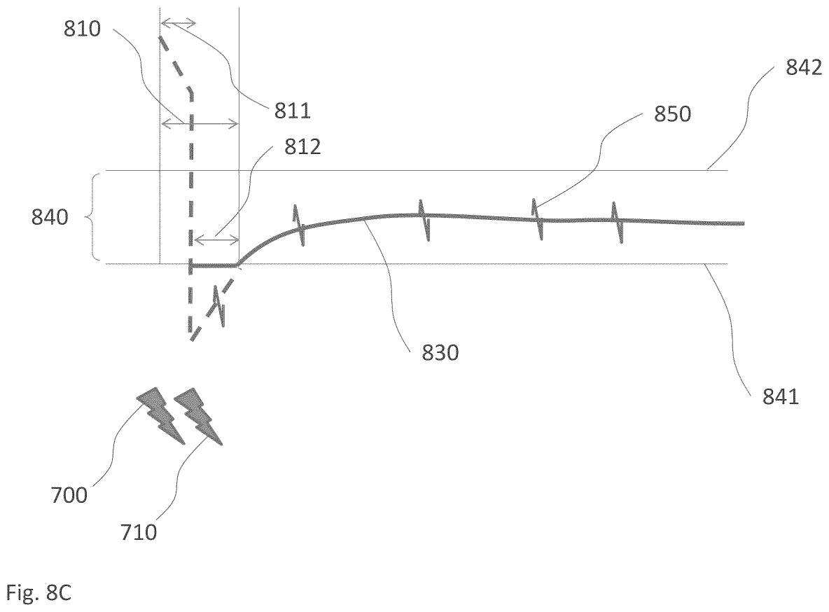

The construction of the implantable medical device 600 does not deviate from the general construction of an implantable medical device according to prior art. However, the way of sensing electric signals from the patient 650 significantly differs and can be achieved in a much shorter time window after having delivered a stimulation pulse than this is possible according to prior art solutions. This will be explained in more detail with reference to FIGS. 7 and 8A to 8C.

FIG. 7 schematically illustrates the delivery of a stimulation pulse 700 and a compensation pulse 710. Both pulses are delivered with the same stimulation unit of an implantable medical device, e.g., with the stimulation unit 610 of the implantable medical device 600 shown in FIG. 6.

The compensation pulse 710 is only generated and delivered with a predetermined temporal delay 720 after having generated the stimulation pulse. This predetermined temporal delay 720 has a relevant physiologic effect in particular in case of an anti-bradycardic therapy. The compensation pulse 710 may not be generated and delivered immediately after the therapeutic stimulation pulse 700 to avoid a reduction of the effect of the stimulation pulse 700. Rather, it is necessary that the stimulation pulse 700 has already induced a propagation of electric signals before the compensation pulse 710 is delivered. In case of stimulating a cardiac muscle, a temporal delay of at least 5 ms is recommended.

The control unit of the implantable medical device (e.g., the control unit 630 of the implantable medical device 600 illustrated in FIG. 6) is arranged and designed to amend an amplitude 711, a duration 712, a net charge, and/or a polarity (in particular of the net charge) of the compensation pulse 710. In addition, the control unit can adjust the temporal delay 720 between the compensation pulse 710 and the stimulation pulse 700. This adjustment of individual parameters of the compensation pulse 710 is particularly performed in dependence on the polarity of the sensed electric signals (i.e., if the sensed electric signals fall below a lower threshold of the dynamic range of the sensing unit or exceed an upper threshold of the dynamic range of the sensing unit).

Generally, the compensation pulse can be an individual pulse or a signal comprising a plurality of pulses. The compensation pulse can also be realized as pulse width modulation (PWM) signal.

FIG. 8A schematically illustrates the effect of a compensation pulse 710 delivered after the stimulation pulse 700. In this and in all other Figures, similar elements will be denoted with the same numeral reference, if appropriate. Thereby, FIG. 8A shows an optimum constellation in which the compensation pulse 710 reduces the post-potential of the stimulation pulse 700 exactly as much as required to bring a neutral line of the sensed electrical signal 830 to lie within the dynamic range 840 of the sensing unit. Then, it is easily possible to detect and evaluate, e.g., a cardiac activity 850 modulated onto the neutral line of the sensed electric signal 830.

By such an optimum compensation of the post-potential of the sensed electric signal 830, the first time period 810, during which the sensed electric signal 830 lies outside the dynamic range 840 of the sensing unit, is significantly shorter than in case of a natural reduction of the post-potential as illustrated in FIG. 5 (cf. the length of the first time period 510 in FIG. 5). Ideally, the neutral line of the sensed electric signal 830 lies approximately in a central area between a lower threshold 841 and an upper threshold 842 of the dynamic range 840. This will make an evaluation of the sensed electric signal 830 particularly easy.

FIG. 8B schematically illustrates a situation in which the compensation pulse 710 is not big enough to fully compensate for the post-potential present on the combined stimulation and sensing electrode due to the previously delivered stimulation pulse 700. Therefore, the first time period 810, during which the sensed electric signal 830 lies outside the dynamic range 840 of the sensing unit, is longer than in case of the situation illustrated in FIG. 8A. Consequently, there remains a longer “blind spot” period, during which no exact evaluation of the sensed electric signal 830 is possible. Rather, the sensed electric signal 830 remains during this extended first period of time 810 at the upper threshold 842 of the dynamic range 840 of the sensing unit. A full evaluation of signals present on the sensed electric signal 830, such as cardiac signals 850, will only be possible after the end of the first time period 810.

FIG. 8C schematically illustrates a situation in which the compensation pulse 710 is too strong so that an over-compensation of the post-potential on the combined stimulating and sensing electrode after a previous stimulation pulse 700 occurs. This also results in an extended first time period 810 during which no proper evaluation of the sensed electric signal 830 is possible since it lies outside the dynamic range 840 of the sensing unit. At the end of a first section 811 of the first time period 810, the sensed electric signal changes its polarity and therewith its sign. Thus, the over-compensation by the compensation pulse 710 results in that the sensed electric signal which lies originally above the upper threshold 842 of the dynamic range 840 becomes too negative so that it still lies outside the dynamic range 840, but below the lower threshold 841 of the dynamic range 840 of the sensing unit. Only after having recovered from this overcompensation during a second section 812 of the first time period 810, it will be possible to fully evaluate the sensed electric signal 830 and individual signals modulated on its neutral line, such as cardiac signals 850.

In order to avoid such an overcompensation as illustrated in FIG. 8C and an insufficient compensation as illustrated in FIG. 8B, but to obtain an optimum (or at least an optimized) situation as illustrated in FIG. 8A, the compensation pulse 710 is typically applied in subsequent compensation rounds in an interactive way. Then, the compensation pulse 710 is increased if it was not yet sufficiently high in a previous compensation round. Furthermore, the compensation pulse is decreased if it was too high in a previous compensation round. As already explained above, such iterative optimization of the compensation pulse 710 can be done, e.g., in steps of 10% increments until an overcompensation as illustrated in FIG. 8C is observed. Then, an average value of the compensation pulse 710 of a couple of previous compensation rounds (e.g., the latest 2 or 3 compensation iterations) is used to compensate post-potentials on the combined stimulation and sensing electrode after a subsequent stimulation pulse 700 in an optimized way.

It is also possible to provide the compensation pulse iteratively after one and the same stimulation pulse, e.g., by providing the compensation pulse in form of a PWM signal. Then, the compensation pulse will provide a charge to reduce the post-potential on the electrode iteratively in small portions. In breaks between individual charge provisions, the success of the compensation pulse can be checked and the compensation can be continued or terminated, depending on the value of the neutral line of the sensed electric signal 830 within the dynamic range 840 of the sensing unit. The partial amounts of the compensation pulse 710 can also be provided with different polarity. As an example, if the partial charge of the compensation pulse 710 resulted in an over-compensation of the post-potential, the next partial amount of the compensation pulse 710 can have an opposite polarity to properly compensate for the previous inadvertent over-compensation.

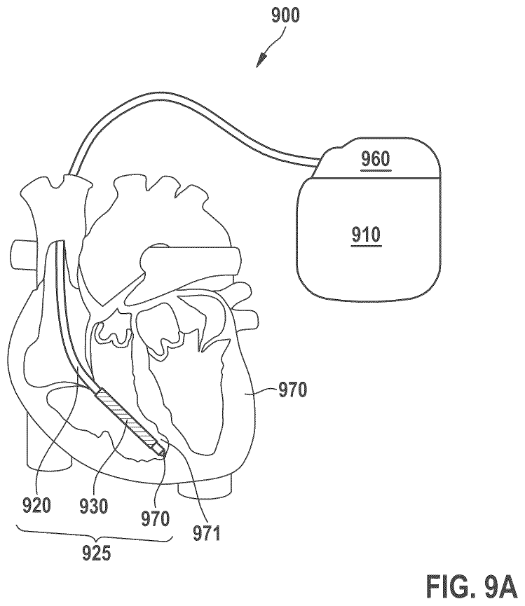

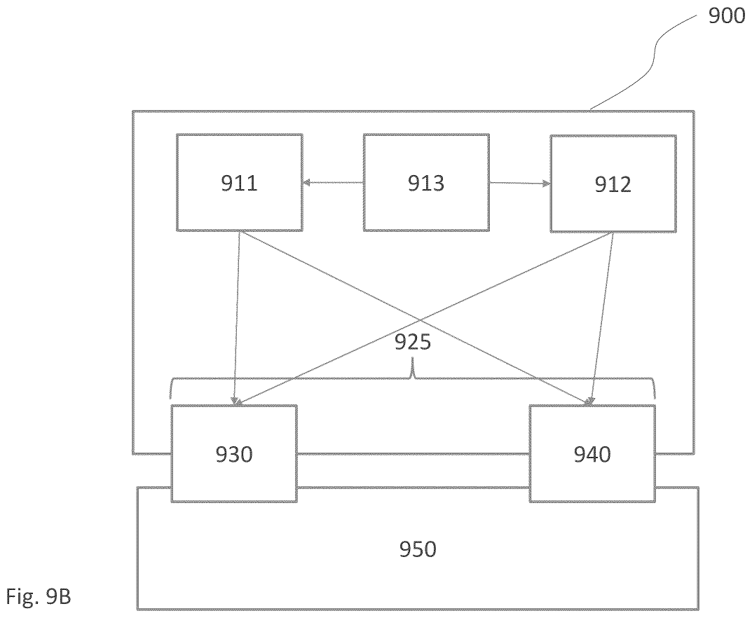

FIG. 9A shows an embodiment of a transvenous ICD 900 comprising a housing 910 and a combined defibrillation and sensing electrode 925. This combined defibrillation and sensing electrode 925 comprises an electrode lead 920 and two electrode poles, namely a shock coil 930 and a tip electrode pole 940. The shock coil 930 and the tip electrode pole 940 are implanted into the right ventricle 971 of a human heart 970. In doing so, stimulation pulses such as defibrillation shocks delivered by the shock coil 930 are directly delivered at the site of action. In addition, the tip electrode pole 940 typically directly contacts cardiac tissue so that it can easily sense cardiac electric signals from the human heart 970. The housing 910 is located outside the human heart 970. The combined defibrillation and sensing electrode 925 is connected to a stimulation unit and a sensing unit contained within the housing 910. For this purpose, the combined defibrillation and sensing electrode 925 enters the housing 910 through a header 960 that realizes the electric connections of the units contained in the housing 910.

FIG. 9B shows a typical setup of an embodiment of a transvenous ICD 900 that can also be denoted as electrotherapeutic implant. The transvenous ICD 900 comprises a stimulation unit 911 and a sensing unit 912. Furthermore, it comprises a control unit 913 that is operatively coupled to the stimulation unit 911 and to the sensing unit 912.

The transvenous ICD 900 furthermore comprises a shock coil 930 serving as first electrode pole and a tip electrode pole 940 serving as second electrode pole that are located on a combined defibrillation and sensing electrode 925. The shock coil 930 and the tip electrode pole 940 get into (electric) contact with the body of a patient 950. The shock coil 930 serves both for delivering a stimulation pulse to the patient 950 as well as for sensing electric signals from the patient 950. In case of such sensing applications, the shock coil 930 serves as counter electrode for the tip electrode pole 940 that is the factual sensing electrode pole.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Implantable cardioverter-defibrillator comprising:
a housing, wherein the housing comprises a stimulation unit and a sensing unit;
a combined defibrillation and sensing electrode for delivering stimulation pulses to a patient in need thereof and for sensing electrocardiogram signals of the same patient, wherein the combined defibrillation and sensing electrode is electrically connected to the stimulation unit and to the sensing unit;
wherein
the combined defibrillation and sensing electrode comprises an electrode pole that serves for delivering stimulation pulses to a patient in need thereof and for sensing electrocardiogram signals of the same patient; and
in that the sensing unit comprises a circuitry enabling the sensing unit to tolerate a post-potential of a stimulation pulse delivered by the combined defibrillation and sensing electrode.

2. Implantable cardioverter-defibrillator according to claim 1, wherein the combined defibrillation and sensing electrode comprises only a single electrode pole that serves for delivering stimulation pulses to a patient in need thereof and for sensing electrocardiogram signals of the same patient.

3. Implantable cardioverter-defibrillator according to claim 1, wherein the electrode pole of the combined defibrillation and sensing electrode is designed as shock coil.

4. Implantable cardioverter-defibrillator according to claim 1, wherein the electrode pole of the combined defibrillation and sensing electrode (is designed isodiametric to an electrode lead body of the combined defibrillation and sensing electrode.

5. Implantable cardioverter-defibrillator according to claim 1, wherein the circuitry provides the sensing unit with a dynamic range that allows the sensing unit to tolerate a post-potential of a stimulation pulse delivered by the combined defibrillation and sensing electrode.

6. Implantable cardioverter-defibrillator according to claim 1, wherein the circuitry is designed and arranged to perform a high-pass filtering of signals received by the combined defibrillation and sensing electrode.

7. Implantable cardioverter-defibrillator according to claim 1, wherein the circuitry serves for a DC offset compensation of signals received by the combined defibrillation and sensing electrode prior to processing the signals by the sensing unit.

8. Implantable cardioverter-defibrillator according to claim 1, wherein an electrode lead of the combined defibrillation and sensing electrode is connected with the stimulation unit and/or with the sensing unit with a unipolar connector.

9. Implantable cardioverter-defibrillator according to claim 1, wherein the implantable cardioverter-defibrillator comprises a communication unit by which the implantable cardioverter-defibrillator can be connected with a home monitoring system in a wireless manner.

10. Implantable cardioverter-defibrillator according to claim 1, wherein the housing has a volume of not more than 70 cm³, in particular not more than 60 cm³.

11. Implantable cardioverter-defibrillator according to claim 1, wherein a header of the housing has a volume of not more than 6 cm³, in particular not more than 5 cm³.

12. Implantable cardioverter-defibrillator according to claim 1, wherein the combined defibrillation and sensing electrode is a non-transvenous, in particular a subcutaneously, a submuscularly and/or a substernally implantable electrode.

13. Method for operating an implantable cardioverter-defibrillator according to claim 1, the method comprising the following steps:
a) receiving, with the electrode pole of the combined defibrillation and sensing electrode, electrocardiogram signals of a patient carrying the implantable cardioverter-defibrillator in an implanted state;
b) preprocessing the received electrocardiogram signals with the circuitry of the sensing unit of the implantable cardioverter-defibrillator, thus enabling the sensing unit to tolerate a post-potential of a stimulation pulse previously delivered by the combined defibrillation and sensing electrode; and
c) processing the preprocessed signals by the sensing unit.

14. Method according to claim 13, wherein preprocessing the received electrocardiogram signals comprises subjecting the received electrocardiogram signals to a high-pass filter.

15. Method according to claim 13, wherein preprocessing the received electrocardiogram signals comprises applying a DC offset compensation.
